(19) United States
(12) Patent Application Publication
(10) Pub. No.: US 2024/0099861 A1
(43) Pub. Date: Mar. 28, 2024

(54) Walking Simulator, in Particular to Test a Prosthetic Device

(71) Applicant: UNIVERSITA' DEGLI STUDI DI PADOVA, Padova (PD) (IT)

(72) Inventors: Nicola Petrone, Noventa Padovana (IT); Paolo Mistretta, Verona (IT)

(21) Appl. No.: 18/550,759

(22) PCT Filed: Mar. 15, 2022
(86) PCT No.: PCT/IB2022/052328

§ 371 Date: Sep. 15, 2023

(30) Foreign Application Priority Data
Mar. 16, 2021 (IT) 102021000006293

(51) Int. Cl. A61F 2/76

(52) U.S. Cl. CPC A61F 2/76; A61F 2002/607; A61F 2002/7695

(57) ABSTRACT

A walking simulator includes: a lower-limb prosthetic device having at least a foot portion, a tibial element, and an ankle articular center suitable to connect the foot portion to the tibial element; a base structure, an arm coupled to the base structure via a coupler that allows the arm to rotate or pivot about a substantially horizontal axis of rotation, wherein the arm includes a fastener for constraining the prosthetic device to the arm so that it extends substantially parallel to the arm; a substantially horizontal base positioned under the prosthetic device, wherein the base includes a top portion suitable to receive in abutment a sole of the foot portion of the prosthetic device; a first actuator associated with the arm for creating a pivoting or oscillating rotary motion of the arm about the axis of rotation and for controlling the leg angle of the prosthetic device constrained to the arm.

### BACKGROUND OF THE INVENTION

### 1. The Field of the Invention

The present invention relates to a walking simulator. In particular, the simulator according to the present invention is adapted for testing a lower-limb prosthetic device, in particular of the transtibial type.

### 2. The Relevant Technology

Several types of lower-limb prosthetic devices, in particular of the transtibial type (also referred to as ankle-foot prostheses), are currently known in the art, which have been conceived to replace a portion of a lower limb of an amputated person. In this regard, it is known that lower-limb prosthetic devices, in particular of the transtibial type, comprise:
a foot portion;
a tibial element or tibia;
an ankle articular center, suitable to connect the foot portion to the tibial element. Said ankle articular center may be provided by a physical joint, e.g., a hinge, positioned between the tibial element and the foot portion, or by the relative motion between the tibial element and the foot portion when the ankle articulation is emulated through the flexibility of elastic components.

In this frame, lower-limb prosthetic devices, in particular of the transtibial type, can be classified into the following configurations, which differ as concerns the type of ankle articulation: with a rigid articulation, with a uniaxial articulation, or with a multiaxial articulation. The configuration with a rigid articulation allows no possibility of relative motion between the foot and the tibial segment at ankle level; the configuration with a uniaxial articulation gives the possibility of rotating the toe in the sagittal plane in dorsiflexion and plantarflexion; the configuration with a multiaxial articulation also allows, in addition to dorsiflexion and plantarflexion, inversion/eversion in the frontal plane.

Furthermore, lower-limb prosthetic devices, in particular of the transtibial type, may be either wholly passive or electronically controlled by microprocessors to adapt the behaviour of the prosthetic device to various contingent situations, thus improving the behaviour of the prosthetic device.

The prosthetic devices currently known in the art pursue a very difficult task, i.e., allowing amputated people to walk with a gait which is as natural and physiological as possible.

However, the complexity of human locomotion is such that the known prosthetic devices normally provide limited functionality compared with a real human leg, so that some requirements cannot be wholly fulfilled.

In this respect, it must also be pointed out that people who have suffered amputation and wearers of lower-limb prosthetic devices establish compensation mechanisms which result in gait asymmetry and which may lead to the onset of secondary disorders (e.g., osteoarthritis and osteoporosis) and/or to higher metabolic energy consumption, inevitably resulting in increased fatigue.

Moreover, the use of the prosthetic devices known in the art typically leads to further problems for the wearers, such as, for example, postural balance and hip-spine alignment problems.

The inherent difficulties in adequately developing such a complex design as that of lower-limb prosthetic devices make it necessary, therefore, to develop suitable testing equipment which can appropriately and realistically simulate human locomotion. In this regard it must be pointed out that, by using such testing equipment, designers can improve the prosthetic device during the early engineering stages and can effectively test the performance of the prosthetic device under various conditions.

The regulations currently in force for testing lower-limb prostheses, in particular those included in the ISO 10328:2016 and ISO 22675:2016 standards, envisage the use of testing methods wherein the prosthesis is subjected to loads that are very different from those which are typical of a physiological gait, and this may lead to incorrect assessment of the prosthesis' performance. It is also worth underlying that such standards have been expressly designed to evaluate prostheses from a structural viewpoint only, neglecting all functional aspects thereof.

In this respect, some tests for functional prosthesis evaluation are included in the ISO TS 16955:2016 technical specifications. However, such tests re-create load conditions that are very different from those which are typical of the physiological gait.

In this regard, it should also be noted that modern prostheses, the performance levels of which are constantly rising, and which implement an ever increasing number of electronically controlled components (e.g., sensors, servo-valves and motors), require more advanced testing methods capable of evaluating not only their structural integrity, but also their functionality under conditions resembling the physiological gait.

Gait testing apparatuses are known in the art which comprise, for example, motion capture systems and force platforms, wherein the participation of human beings is necessary in order to test the prosthetic device.

Such apparatuses permit reproducing the gait in a rather realistic manner, but they still have some drawbacks. In fact, the involvement of human beings in the assessment of the performance of the lower-limb prosthetic device makes it impossible to obtain an objective, certain, ethical, repeatable, controllable and quantitative testing method.

As a consequence of this, human gait simulators are also known in the art which have been conceived for testing and assessing the performance of a prosthetic device without requiring human participation.

Nevertheless, the simulators known in the art have been conceived in such a way that the performance levels required from the actuators included therein cannot be kept relatively low, particularly in terms of force, stroke and speed, and this is inevitably reflected in excessively high costs of the whole simulator.

In particular, in order to be able to simultaneously reproduce several degrees of freedom, the known simulators are equipped with actuators that are typically arranged in series, resulting in considerable inertial forces deriving from the substantial masses in motion; in this regard, it must also be pointed out that, due to the speed of the walking movement, inertial forces are typically a preponderant factor when dimensioning human walking simulators.

### SUMMARY OF THE INVENTION

In this frame, it is the main object of the present invention to provide a walking simulator, in particular for testing a prosthetic device, which has been so conceived as to overcome the drawbacks of the prior art.

In particular, it is one object of the present invention to provide a walking simulator, in particular for testing a prosthetic device, wherein said simulator is so constructed as to be able to adequately test prosthetic devices without involving human subjects for the purpose of evaluating such prosthetic devices.

It is another object of the present invention to provide a walking simulator, in particular for testing a prosthetic device, wherein said simulator has been so conceived as to allow the performance levels, in terms of force, stroke and speed, of the actuators included therein to be kept relatively low.

It is a further object of the present invention to provide a walking simulator that is not too costly to manufacture and difficult to set up.

It is yet another object of the present invention to provide a walking simulator, in particular for testing a prosthetic device, wherein said simulator is so constructed as to permit obtaining a mechanical behaviour which can be considered to be similar to that of the biological gait, particularly as concerns the biological ankle-foot system.

### BRIEF DESCRIPTION OF THE DRAWINGS

Further objects, features and advantages of the present invention will become apparent in the light of the following detailed description and the annexed drawings, which are provided herein merely by way of non-limiting explanatory example, wherein:

FIGS. 1A and 1B show first and second perspective views, respectively, of a walking simulator, in particular for testing a prosthetic device, according to the present invention;

FIGS. 2A to 2D are schematic views of different operating phases of some components of the walking simulator according to the present invention;

FIGS. 3A and 3B are further schematic views of some components of the walking simulator according to the present invention;

FIGS. 4A and 4B are schematic views of first and second variants, respectively, of the walking simulator according to the present invention;

FIGS. 5A to 5C show, respectively, a top view, a side view and a perspective view of some components of the walking simulator according to the present invention;

FIGS. 6A and 6B are schematic views of different operating phases of the components shown in FIGS. 5A to 5C;

FIG. 7 is an exemplifying diagram concerning the operation of the components shown in FIGS. 5A to 6B;

FIG. 8 is a block diagram that illustrates the operation of a control unit of the human walking simulator according to the present invention.

### DETAILED DESCRIPTION OF THE PREFERRED EMBODIMENTS

Referring now to the annexed drawings, reference numeral 1 designates as a whole a walking simulator according to the present invention.

In accordance with the present invention, the simulator 1 comprises a prosthetic device 2, in particular of the transtibial type, comprising at least a foot portion 2A having a sole 2AP, a tibial element (or tibia) 2B, and an ankle articular center 2C suitable to connect the foot portion 2A to the tibial element 2B. In a preferred embodiment, the prosthetic device 2 is of the transtibial type; however, the teachings of the present invention may also apply to different types of prosthetic devices 2.

As far as the ankle articular center 2C is concerned, it should be noted that (as already explained with reference to known prosthetic devices) it may consist of a physical articular center (such as a joint, e.g., consisting of a hinge) positioned between the foot portion 2A and the tibial element 2B, or it may be provided by relative motion between the foot portion 2A and the tibial element 2B when the prosthetic device 2 is equipped with a virtual, as opposed to physical, ankle articulation center 2C, i.e., emulated through the flexibility of elastic components (in this regard, reference should be made to those prosthetic devices which exploit their intrinsic flexibility in order to mimic or emulate the ankle angle without having a physical articulation, e.g., prosthetic devices made of carbon fiber).

The simulator 1 according to the present invention comprises a base structure 3 and an arm 4 coupled to the base structure 3 via coupling means 5, 5A that allow said arm 4 to rotate or pivot about a substantially horizontal axis of rotation A. Moreover, the arm 4 comprises fastening means 4A for constraining the prosthetic device 2 to said arm 4 so that it extends substantially parallel to said arm 4.

In a preferred embodiment, said fastening means 4A comprise a pyramid-shaped fitting known in the art.

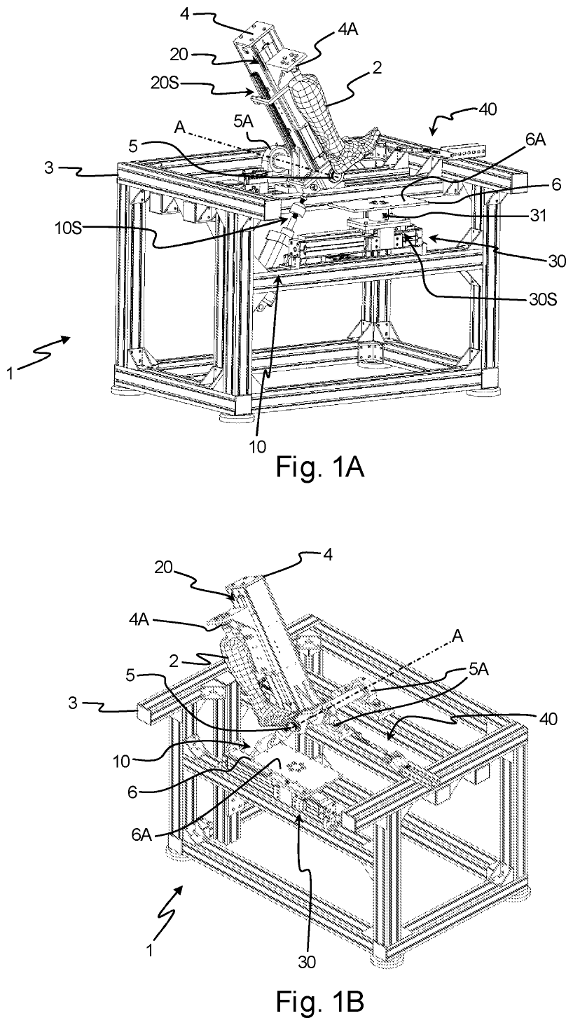

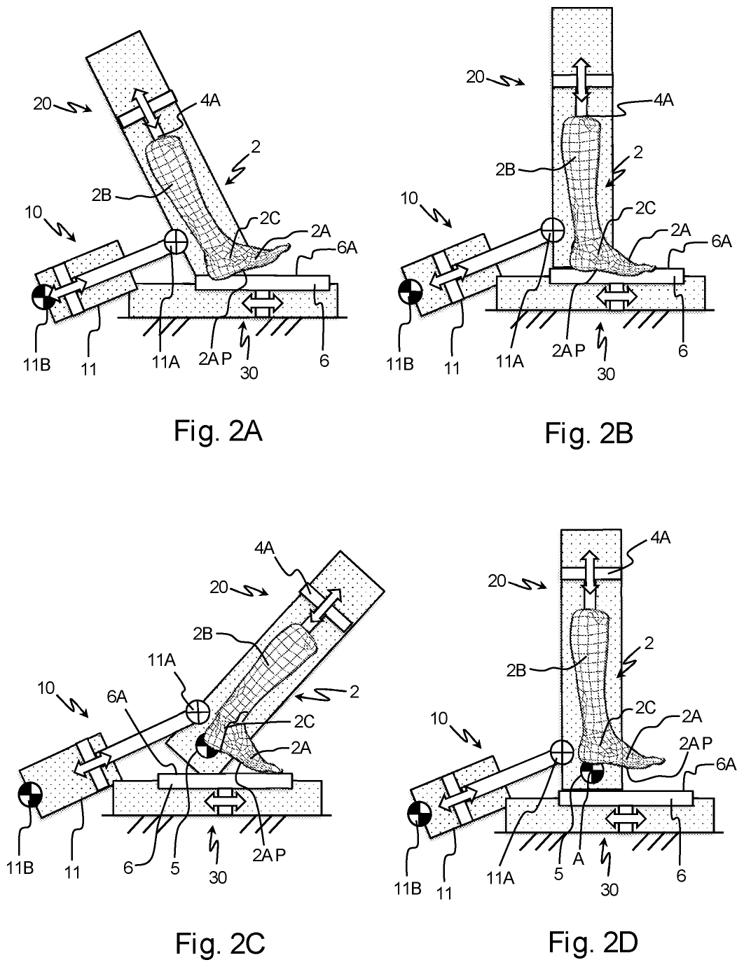

The mutual positioning of the prosthetic device 2 and the arm 4 is visible in FIGS. 1A to 2D, wherein it can be observed that the fastening means 4A are so designed that the prosthetic device 2 is offset relative to a substantially vertical lateral surface of the arm 4, with the tibial element 2B extending substantially parallel to said lateral surface of the arm 4. For example, said fastening means 4A may be implemented as a bracket extending substantially horizontal from a lateral surface of the arm 4, wherein the pyramid-shaped fitting is coupled to an underside of said bracket.

FIGS. 1A and 1B also show that the base structure 3 preferably comprises a modular structure, in particular of the type comprising beams (preferably made of aluminium or steel) provided with tracks that allow adjusting the position of brackets (e.g., consisting of angular members) adapted to mutually connect said beams. Such a construction makes it possible to easily and effectively adjust the position of the various components of the simulator 1 according to the present invention, in particular by adjusting the relative positions of the beams forming the base structure 3.

It is however clear that the base structure 3 and/or the fastening means 4A may also be constructed otherwise.

Furthermore, the coupling means 5, 5A that allow said arm 4 to rotate or pivot about (i.e., relative to) said horizontal axis of rotation A preferably comprise a shaft 5 rigidly constrained to a lower end of the arm 4 and suitable to rotate about an axis of rotation substantially coinciding with said substantially horizontal axis of rotation A; therefore, in the present description reference will be made to the fact that both the arm 4 and the shaft 5 can rotate about said axis of rotation A.

Said coupling means 5, 5A also comprise at least one connection element 5A suitable to couple said shaft 5 to the base structure 3, said at least one connection element 5A being so constructed as to allow the shaft 5 to rotate about (relative to) said axis of rotation A.

In the embodiment shown in FIGS. 1A and 1B, said connection means 5A comprise at least one hinge fastened to the base structure 3 and preferably provided with one or more bearings suitable to allow the shaft 5 to rotate about the axis of rotation A.

In this case as well, it is clear that the coupling means 5, 5A may also be implemented differently than shown in the annexed figures, provided that such coupling means 5, 5A are so constructed as to allow the arm 4 (and hence the shaft 5 rigidly constrained to the arm 4) to pivot or rotate about said substantially horizontal axis of rotation A.

The simulator 1 further comprises a substantially horizontal (i.e., substantially perpendicular to the arm 4) base 6 positioned under the prosthetic device 2, wherein said base 6 comprises a top portion 6A suitable to receive in abutment the sole 2AP of the foot portion 2A of said prosthetic device 2.

In this respect, as can be seen in the annexed drawings, the upper portion 6A of the base 6 consists of the surface of the base 6 that is suitable to receive in abutment the sole 2AP of the foot portion 2A, i.e., that surface of the base 6 which faces upwards and towards the prosthetic device 2. It is therefore apparent that the base 6 is designed to simulate a ground whereon walking occurs.

The simulator 1 according to the present invention further comprises first actuating means 10, 10′, 10″ associated (whether directly or indirectly, e.g., via suitable links) with the arm 4 for creating a horizontally pivoting or oscillating rotary motion of the arm 4 (and hence of the shaft 5 to which said arm 4 is solidly connected) about the axis of rotation A and for controlling the leg angle of the prosthetic device 2 constrained to said arm 4, i.e., for reproducing the sagittal leg angle of a walking person.

In the embodiment shown in FIGS. 1A to 3, 6A and 6B, said first actuating means (designated as a whole by reference numeral 10) comprise a linear actuator 11 connected to the arm 4 and to the base structure 3 via a first hinge 11A and a second hinge 11B, respectively. Preferably, in such an embodiment the linear actuator 11 consists of a cylinder-piston mechanism, in particular of the servo-pneumatic type; moreover, the first hinge 11A is movable and the second hinge 11B is fixed relative to the structure 3.

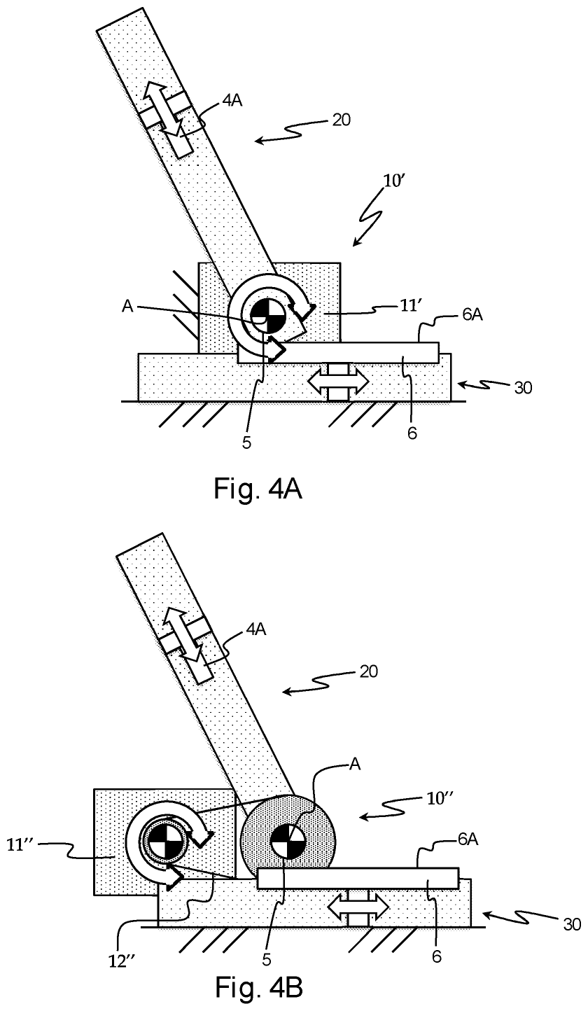

FIG. 4A is a schematic view of a first variant of the human walking simulator 1 according to the present invention; in such first variant, the first actuating means (designated as a whole by reference numeral 10′ in FIG. 4A) comprise a rotary actuator 11′ connected to the shaft 5; preferably, in such an embodiment the rotary actuator 11′ is of the servo-pneumatic, servo-hydraulic or servo-electric type.

In a second variant of the human walking simulator 1, shown in FIG. 4B, said first actuating means (designated as a whole by reference numeral 10″ in FIG. 4B) comprise a rotary actuator 11″ connected to the shaft 5 by means of a transmission system 12″, in particular of the reduction gear type; in the embodiment shown in FIG. 4B, the transmission system 12″ is of the chain or belt type, but clearly it may also be of the gear train type.

The simulator 1 according to the present invention comprises first sensor means (designated as a whole by reference 10S in FIGS. 1A and 1B) associated with the first actuating means 10, 10′, 10″ for controlling the position and/or force of said first actuating means 10, 10′, 10″.

Preferably, in the embodiment shown in FIGS. 1A to 3, 6A and 6B, said first sensor means 10S comprise at least one position sensor and at least one force sensor; in particular, in such an embodiment said at least one position sensor comprises a magnetostrictive sensor and said at least one force sensor comprises a uniaxial cell.

As far as the variants shown in FIGS. 4A and 4B are concerned, the angle of the arm 4 (and hence of the shaft 5, which is rigidly constrained to the arm 4) is preferably calculated on the basis of the dimensions of the kinematic mechanism of the first actuating means 10′, 10″ and the position sensor of said first actuating means 10′, 10″.

Alternatively, in such variants the simulator 1 may comprise first sensor means 10S which may comprise a sensor (e.g., a potentiometric sensor or a magnetic encoder or another type of sensor known in the art) capable of sensing the angular position of the shaft 5 relative to the vertical of the arm 4.

The simulator 1 further comprises second actuating means (designated as a whole by reference numeral 20 in the annexed drawings) associated (whether directly or indirectly) with the fastening means 4A for permitting a linear translational motion of the prosthetic device 2 along the arm 4. By way of example, this translational motion may allow reproducing, by closed-loop control, a dynamic or kinematic quantity variable with such degree of freedom, e.g., the vertical ground reaction force or the vertical translation of the ankle articular center 2C.

The simulator 1 according to the present invention comprises second sensor means (designated as a whole by reference numeral 20S in FIG. 1A) associated with the arm 4 and/or with the second actuating means 20 for controlling the position of said second actuating means 20. Preferably, said second sensor means 20S comprise at least one position sensor, in particular of the magnetostrictive type.

The simulator 1 further comprises third actuating means (designated as a whole by reference numeral 30 in the annexed drawings) associated with the base 6 for permitting a linear translational motion of said base 6 along a substantially horizontal plane (as is particularly visible in FIGS. 2A to 4B, 6A and 6B). By way of example, this translational motion may allow reproducing, by closed-loop control, a dynamic or kinematic quantity variable with such degree of freedom, e.g., the anteroposterior ground reaction force or the anteroposterior translation of the ankle articular center 2C.

In a preferred embodiment, said second actuating means 20 and said third actuating means 30 comprise each at least one linear actuator, in particular of the servo-pneumatic rodless type. The choice of this type of actuators is due to the fact that they can withstand high off-axis loads because of the presence of a carriage integrated with the mobile part of the actuator itself; in addition, such actuators are also relatively inexpensive and light.

As an alternative, said second actuating means 20 and third actuating means 30 may be so implemented as to comprise alternative actuation systems having similar properties in terms of force, speed, stroke and off-axis load capacity, such as, for example, a combination of linear actuators (e.g., pneumatic linear rod-type, electric linear or electric rotary actuators coupled to a screw and nut system) and associated guides (e.g., carriages or linear guides).

The simulator 1 according to the present invention comprises third sensor means (designated as a whole by reference numeral 30S in FIGS. 1A and 1B) associated with the base 6 and/or with the third actuating means 30 for controlling the position of said base 6 and the vertical and anteroposterior reaction force exerted by the prosthetic device 2 on the base 6.

Preferably, said third sensor means 30S comprise at least one position sensor, in particular of the magnetostrictive type, for controlling the position of said base 6.

In addition, said third sensor means 30S comprise a load cell 31, in particular a six-axis one, for controlling the vertical and anteroposterior reaction force exerted by the prosthetic device 2 on the base 6. When a six-axis load cell 31 is implemented, it is possible to measure all six force components (three forces and three torques) in the x, y, z directions; highly detailed analyses can thus be made, e.g., the analysis of the pressure center trajectory or the analysis of the medial-lateral forces.

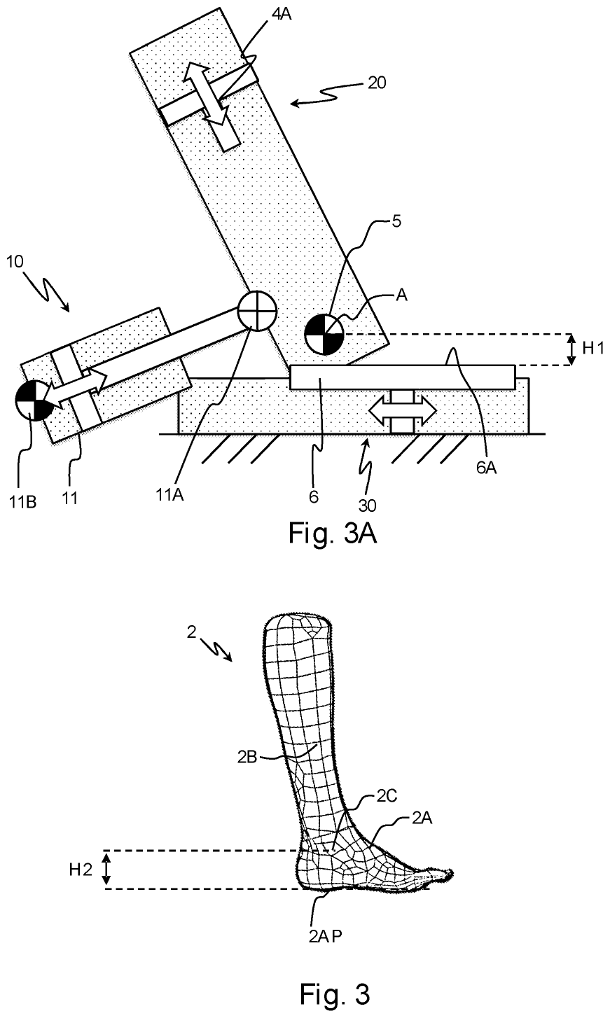

FIG. 3A shows the shaft 5, the base 6 and the actuating means 10, 20, 30, while FIG. 3B shows the prosthetic device 2.

According to the present invention, as is particularly noticeable in said FIGS. 3A and 3B, said shaft 5 is integral with a lower end of the arm 4, wherein the axis of rotation A of the shaft 5 and the upper portion 6A of the base 6 are mutually positioned in the sagittal plane (not shown in the annexed drawings, since it is known in the art) at a first height H1 substantially corresponding to a second height H2 (still referred to the sagittal plane) of the ankle articular center 2C of the prosthetic device 2. In substance, said second height H2 corresponds to the distance in the sagittal plane between the sole 2AP of the foot portion 2A and the ankle articular center 2C.

In accordance with the present invention, said first height H1 between the axis of rotation A (of the arm 4 and the shaft 5) and the upper portion 6A of the base 6 is comprised between 45 mm and 85 mm.

In a preferred embodiment, said first height H1 between the axis of rotation A and the upper portion 6A of the base 6 is approximately 70 mm.

Indicatively, said first height H1 (in the sagittal plane, in the vertical direction) between the axis of rotation A and the upper portion 6A of the base 6 corresponds to approximately 0.04 times the height of a user of the prosthetic device 2, i.e., a user whose gait is to be reproduced by means of the simulator 1 according to the present invention.

From the annexed figures and the present description, it clearly emerges that the assembly consisting of the arm 4 and the shaft 5 is shaped as an upside-down simple pendulum, wherein the oscillating (or horizontally pivoting) rotary motion of the arm 4 (and hence of the shaft 5 to which said arm 4 is solidly connected) about the axis of rotation A is produced by the first actuating means 10, 10′, 10″.

The peculiar features of the present invention make it possible to correctly position the fixed axis of rotation A of the rotary arm 4 vertically in the sagittal plane, wherein said axis of rotation A must be positioned as close as possible to the projection in the sagittal plane of the instantaneous rotation center of the prosthetic device 2 throughout the duration of the movement to be reproduced on the simulator 1 of the present invention. This minimizes the stroke required from the actuating means 10, 10′, 10″, 20, 30, due to the fact that a flat motion (i.e., the motion of the leg/prosthetic device 2 in the sagittal plane) is comparable to a rotation relative to the instantaneous rotation center without any additional translations (produced by the second and third actuating means 20, 30 in the walking simulator 1 according to the present invention). While walking, the instantaneous rotation center of the prosthetic device 2 according to the present invention (which corresponds to that of a biological leg) in the sagittal plane describes a trajectory over time; however, during the first part of the gait cycle (i.e., indicatively between 0% and 45% of the swing phase and between 0% and 80% of the stance phase), such trajectory remains almost stationary and, in addition, close to the ankle articular center 2C. Because of this, by positioning the axis of rotation A of the rotary arm 4 at the ankle articular center 2C during the first part of the gait stance phase it is possible to minimize the translation required from the second and third actuating means 20, 30. This permits keeping the performance levels required from the actuators employed in the simulator 1 relatively low in terms of force, stroke and speed, so that low-cost and easy-to-tune actuators can be used.

At the same time, the peculiar provisions of the present invention make it possible to realize the simulator 1 in such a way as to obtain a mechanical behaviour that is similar to that of the biological gait, particularly as concerns the tibia (leg) angle and the vertical and anteroposterior ground reaction forces.

On account of the fact that the rotary arm 4 may have a considerable mass, said arm 4 accumulates much kinetic energy when it is moved in order to simulate the gait.

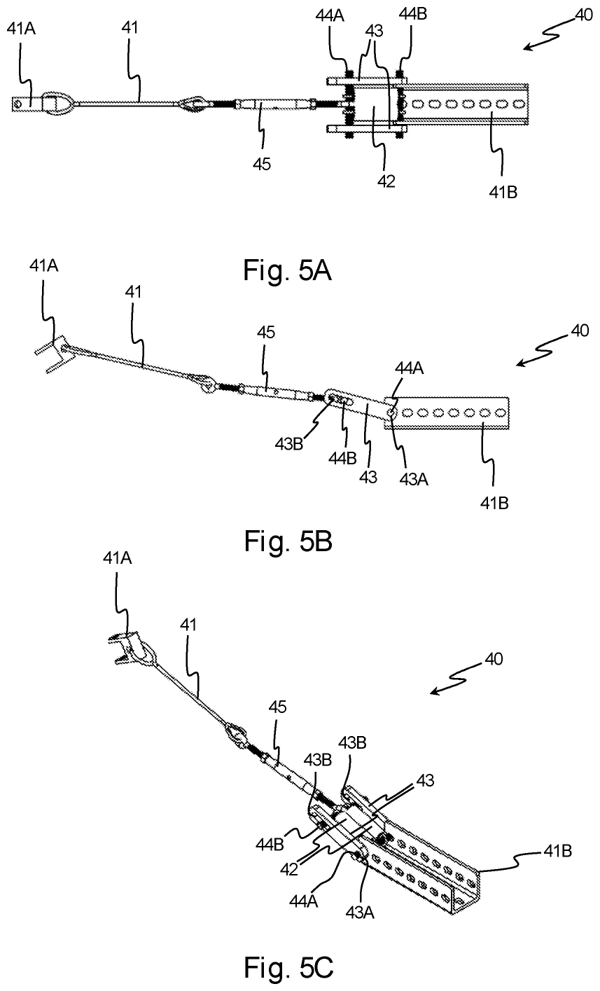

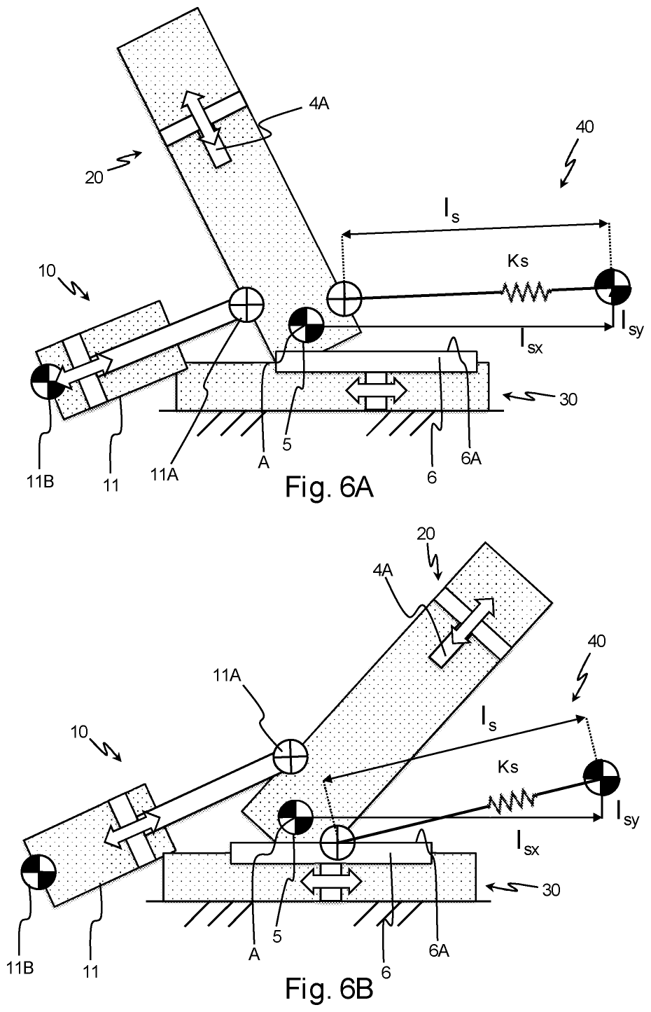

As can be observed in FIGS. 1A, 1B and 5A to 6B, in accordance with the present invention the simulator 1 comprises a safety system (designated as a whole by reference numeral 40) for preventing the arm 4 from travelling past the limits of the positive leg angle and negative leg angle during its oscillating (or horizontally pivoting) rotary motion about the axis of rotation A; in particular, FIG. 6A refers to the bottom dead center of the first actuating means 10, while FIG. 6B refers to the top dead center of the first actuating means 10.

In accordance with a preferred embodiment, said safety system 40 comprises a substantially (ideally) inextensible rope 41 having a first end 41A secured to the arm 4 and a second end 41B secured to the base structure 3. In the illustrative embodiment shown in the annexed figures, the first end 41A consists of a U-shaped bracket, whereas the second end 41B consists of a U channel.

In addition, the safety system 40 comprises at least one elastic element 42 (e.g., a coil spring or a pair of parallel coil springs) interposed between the rope 41 and at least one of said ends 41A, 41B.

In particular, the safety system 40 comprises a pair of parallel plates 43 adapted to connect said at least one elastic element 42 to the rope 41 and to one of said ends 41A, 41B, wherein each plate 43 comprises:
a hole 43A suitable to receive a first bar 44A associated with a first end of the elastic element 42;
a slotted hole 43B suitable to receive a second bar 44B associated with a second end of the elastic element 42,
and wherein the slotted holes 43B of the plates 43 constitute the rigid stopper of the arm 4.

In a preferred embodiment, the safety system 40 comprises an adjustable-length tensioner 45, in particular positioned between the rope 41 and said at least one elastic element 42, for adjusting the angles of engagement of said at least one elastic element 42.

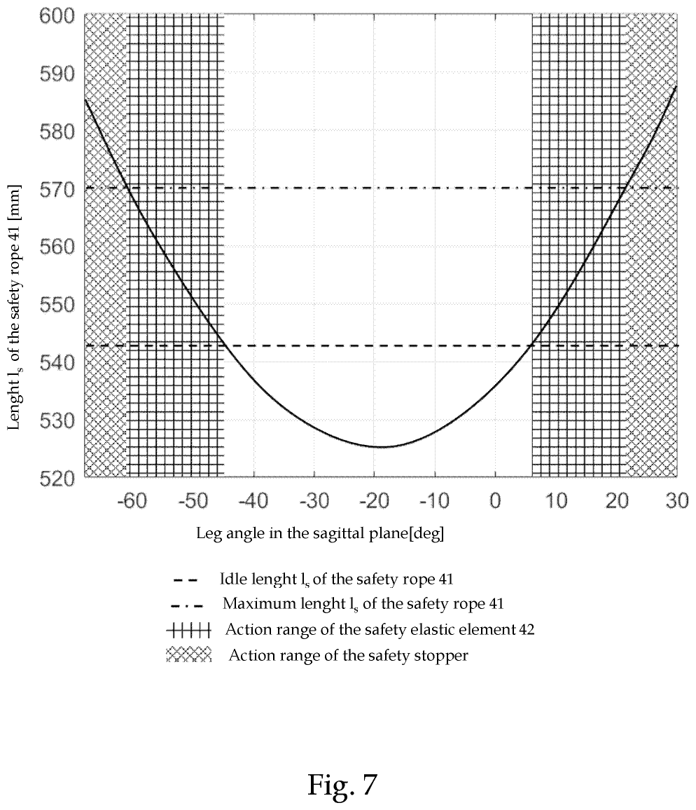

FIG. 7 shows an exemplifying diagram concerning the operation of the safety system 40 according to the present invention.

The length lₛ of the safety rope 41 varies with the leg angle, and hence with the angle of the rotary arm 4. When such length lₛ is greater than the idle length, said at least one elastic element 42 will extend, generating a force that will tend to cause the rotary arm 4 to return towards the minimum region of the curve of the length lₛ. When the length lₛ exceeds a given critical value, a rigid stopper will come into play, rigidly preventing any further extension beyond the minimum region of the curve.

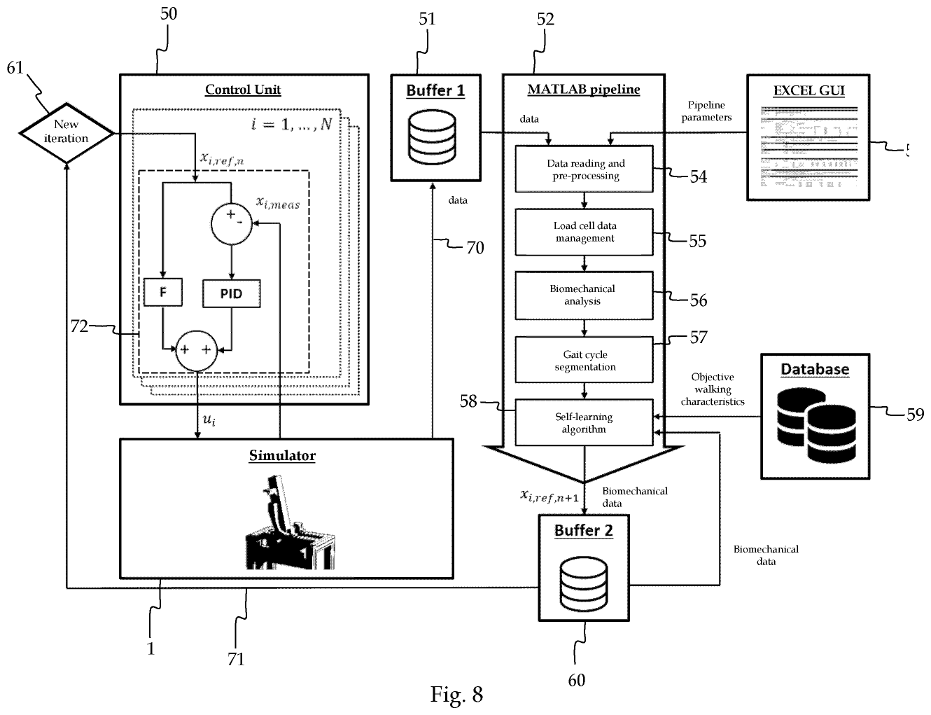

With reference to FIG. 8, there is shown a block diagram that illustrates the operation of a control unit 50 of the human walking simulator 1 according to the present invention.

The process starts with a first walking simulation (block 61, “new iteration”), carried out in the control unit 50 by using an attempt command signal x_{i,ref,n} and by detecting the signals x_{i,meas} coming from the simulator 1, in particular coming from the sensor means 10S,20S,30S.

In this regard, it should be noted that the number N of analyzed signals will be N=3 when testing a passive prosthetic device 2, and N=3+n when testing a prosthetic device 2 equipped with an articulation (in the case of a uniaxial prosthetic device 2) or a plurality of articulations (in the case of a multiaxial prosthetic device 2), wherein said prosthetic device 2 comprises a number n of additional actuating means associated with respective sensor means.

The control unit 50 generates command signals uᵢ, in particular using a standard F-PID (feed-forward+proportional, integral and derivative) architecture 72.

The command signals uᵢ are suitable to control the position of all of the N actuating means 10, 10′, 10″, 20, 30 available.

After having carried out the walking simulation with the actuators positioned in accordance with the command signals uᵢ the data of the various sensor means 10S, 20S, 30S are inputted to a first data buffer 51 over a first data bus 70 for the next analysis phase.

The first data buffer 51 is fed, along with pipeline parameters, to a pipeline 52.

The pipeline parameters used by the pipeline 52 are automatically read from a table, e.g., an Excel sheet, so as to facilitate the management of such parameters.

The pipeline 52 automatically carries out a biomechanical analysis on all data obtained during the walking simulation and, after comparing the results with an actual gait database 59, generates new attempt command signals x_{i,ref,n+1} for all of the N actuating means 10, 10′, 10″, 20, 30 available.

More in detail, the pipeline 52 comprises a plurality of software modules 54,55,56,57,58, e.g., created in the Matlab language, that provide a set of functions:
a first module 54 reads and pre-processes the data coming from the first data buffer 51 and the pipeline parameters;
a second module 55 processes the data coming from at least one load cell 31;
a third module 56 makes a biomechanical analysis of the simulated walk;
a fourth module 57 segments the gait cycle of the simulated walk;
a fifth module 58 comprises a self-learning algorithm that receives, as input, the output of the fourth module 57 and parameters associated with objective walking characteristics contained in a database 59, the output of such algorithm comprising biomechanical data and a signal x_{i,ref,n+1} that are sent to a second buffer 60, which is inputted to the control unit 50 over a second data bus 71.

The process is iterated until convergence is achieved, at least within a predetermined threshold, between the signals outputted by the pipeline 52 and the target ones contained in the database 59.

The software modules 54,55,56,57,58 constitute at least part of a computer program product which can be loaded into a memory associated with a computer.

The features of the human walking simulator 1 according to the present invention, as well as the advantages thereof, are apparent from the above description.

In fact, the peculiar features of the present invention make it possible to correctly position the fixed axis of rotation A of the rotary arm 4 vertically in the sagittal plane, wherein said axis of rotation A (of the arm 4 and of the shaft 5) must be positioned as close as possible to the projection in the sagittal plane of the instantaneous rotation center of the prosthetic device 2 throughout the duration of the movement to be reproduced on the simulator 1 of the present invention. This minimizes the stroke required from the actuating means 10, 10′, 10″, 20, 30, due to the fact that a flat motion (i.e., the motion of the leg/prosthetic device 2 in the sagittal plane) is comparable to a rotation relative to the instantaneous rotation center without any additional translations (produced by the second and third actuating means 20, 30 in the walking simulator 1 according to the present invention). While walking, the instantaneous rotation center of the prosthetic device 2 according to the present invention (which corresponds to that of a biological leg) in the sagittal plane describes a trajectory over time; however, during the first part of the gait cycle (i.e., indicatively between 0% and 45% of the swing phase and between 0% and 80% of the stance phase), such trajectory remains almost stationary and also close to the ankle articular center 2C. Because of this, by positioning the axis of rotation A of the rotary arm 4 (and also of the shaft 5) at the ankle articular center 2C during the first part of the gait stance phase it is possible to minimize the translation required from the second and third actuating means 20, 30. This permits keeping the performance levels required from the actuators employed in the simulator 1 relatively low in terms of force, stroke and speed, so that low-cost and easy-to-tune actuators can be used.

At the same time, the peculiar provisions of the present invention make it possible to realize the simulator 1 in such a way as to obtain a mechanical behaviour that is similar to that of the biological gait, particularly as concerns the tibia (leg) angle and the vertical and anteroposterior ground reaction forces.

A further advantage of the present invention lies in the fact that the peculiar provisions of the safety system 40 conveniently make it possible to prevent the arm 4 from exceeding the angular stroke limits of the positive leg angle and negative leg angle during its oscillating rotary motion about the axis of rotation A.

It is therefore apparent that the simulator 1 according to the present invention has been so conceived as to prevent the walking simulator 1 from suffering any damage and hurting any operators standing close the simulator 1 in operation.

Moreover, the peculiar provisions of the safety system 40 make it possible to independently define the angles of engagement of said safety system 40 in the two directions of rotation (clockwise and counterclockwise) of the assembly consisting of the arm 4 and the prosthetic device 2. It is therefore apparent that such provisions allow, in particular, the use of a single safety system 40 for both directions of rotation.

By providing the simulator 1 with sensor means 10S, 20S, 30S it is possible to exactly quantify the potential influence of individual factors on a specific quantity of interest and to make an accurate biomechanical analysis of the prosthetic device 2; in addition, based on a comparison between the obtained results and an actual gait database, the control unit of the simulator 1 according to the present invention can have the measured signals converge with target ones.

It is therefore apparent that the simulator 1 according to the present invention has been conceived to simulate a human gait in a highly realistic manner.

It should also be noted that the simulator 1 according to the present invention also proves to be adequate for testing footwear, since it will be sufficient to put a shoe to be tested on the prosthetic device 2 constrained to the arm 4.

The human walking simulator 1 described herein by way of example may be subject to many possible variations without however departing from the novelty spirit of the inventive idea; it is also clear that in the practical implementation of the invention the illustrated details may have different shapes or be replaced with other technically equivalent elements.

It can therefore be easily understood that the present invention is not limited to the above-described human walking simulator 1, but may be subject to many modifications, improvements or replacements of equivalent parts and elements without departing from the inventive idea, as clearly specified in the following claims.

1. A walking simulator comprising:
a lower-limb prosthetic device comprising at least a foot portion having a sole, a tibial element, and an ankle articular center suitable to connect the foot portion to the tibial element;
a base structure,
an arm coupled to the base structure via coupling means that allow said arm to rotate or pivot about a substantially horizontal axis of rotation, wherein said arm comprises fastening means for constraining the prosthetic device to said arm so that it extends substantially parallel to said arm;
a substantially horizontal base positioned under the prosthetic device, wherein said base comprises a top portion suitable to receive in abutment a sole of the foot portion of said prosthetic device;
first actuating means associated with the arm for creating a horizontally pivoting or oscillating rotary motion of the arm about the axis of rotation and for controlling the leg angle of the prosthetic device constrained to said arm;
second actuating means associated with the fastening means for permitting a linear translational motion of the prosthetic device along the arm;
third actuating means associated with the base for permitting a linear translational motion of said base along a substantially horizontal plane,
wherein said axis of rotation and the top portion of the base are mutually positioned in the sagittal plane at a first height comprised between 45 mm and 85 mm.

2. The simulator according to claim 1, wherein said first height and the top portion of the base is approximately 70 mm.

3. The simulator according to claim 1, wherein said first height substantially corresponds to a second height of the ankle articular center of the prosthetic device.

4. The simulator according to claim 1, wherein said coupling means comprise a shaft rigidly constrained to a lower end of the arm and suitable to rotate about an axis of rotation substantially coinciding with said substantially horizontal axis of rotation.

5. The simulator according to claim 4, wherein said coupling means comprise at least one connection element suitable to couple said shaft to the base structure so as to allow the arm and the shaft to rotate about said axis of rotation.

6. The simulator according to claim 1, wherein said first actuating means comprise a linear actuator connected to the arm and to the base structure via a first hinge and a second hinge, respectively.

7. The simulator according to claim 6, wherein said linear actuator consists of a cylinder-piston mechanism.

8. The simulator according to claim 1, wherein said first actuating means comprise a rotary actuator connected to the shaft.

9. The simulator according to claim 1, wherein said first actuating means comprise a rotary actuator connected to the shaft by means of a transmission system.

10. The simulator according to claim 1, wherein said second actuating means and said third actuating means comprise each at least one linear actuator.

11. The simulator according to claim 1, comprising a safety system for preventing the arm from travelling past the limits of the positive leg angle and negative leg angle during its oscillating rotary motion about the axis of rotation.

12. The simulator according to claim 11, wherein said safety system comprises:
a substantially inextensible rope having a first end secured to the arm and a second end secured to the base structure;
at least one elastic element interposed between the rope and at least one of said ends.

13. The simulator according to claim 11, wherein said safety system comprises a pair of plates adapted to connect said at least one elastic element to the rope and to one of said ends, wherein each plate comprises:
a hole adapted to receive a first bar associated with a first end of the elastic element;
a slotted hole suitable to receive a second bar associated with a second end of the elastic element,
and wherein the slotted holes of the plates constitute the rigid stopper of the arm.

14. The simulator according to claim 12, wherein said safety system comprises an adjustable-length tensioner for adjusting the angles of engagement of said at least one elastic element.

15. The simulator according to claim 1, comprising first sensor means associated with the first actuating means for controlling the position and/or force of said first actuating means.

16. The simulator according to claim 15, wherein said first sensor means comprise at least one position sensor and at least one force sensor.

17. The simulator according to claim 1, comprising second sensor means associated with the arm and/or with the second actuating means for controlling the position of said second actuating means.

18. The simulator according to claim 1, comprising third sensor means associated with the base and/or with the third actuating means for controlling the position of said base and the vertical and anteroposterior reaction force exerted by the prosthetic device on the base.

19. The simulator according to claim 18, wherein said third sensor means comprise at least one position sensor for controlling the position of said base.

20. The simulator according to claim 18, wherein said third sensor means comprise a load cell for controlling the vertical and anteroposterior reaction force exerted by the prosthetic device on the base.

21. The simulator according to claim 1, comprising a control unit adapted to receive, as input, an attempt command signal (x_{i,ref,n}) and signals coming from said sensor means, and suitable to generate command signals for driving said actuating means.

22. Method A method of operation of a simulator according to claim 1, wherein said control unit is associated with a pipeline comprising a plurality of software modules,
said method including the following steps:
reading and pre-processing the data coming from the sensor means by means of a first module;
processing the data coming from at least one load cell by means of a second module;
making a biomechanical analysis of the simulated walk by means of a third module;
segmenting the gait cycle of the simulated walk by means of a fourth module;
receiving, as input, the output of the fourth module and parameters associated with objective walking characteristics contained in a database, by means of a fifth module comprising a self-learning algorithm outputting biomechanical data and a signal (x_{i,ref,n+1}) which feeds a second buffer and is inputted to the control unit.

23. A computer program product which can be loaded into a memory associated with a control unit adapted to implement the method according to claim 22.
